(19) United States
(12) Patent Application Publication
(10) Pub. No.: US 2016/0228668 A1
(43) Pub. Date: Aug. 11, 2016

(54) RESPIRATION INSERT FOR INDUCTION MASK

(71) Applicant: Kristy Rowberry Martin, Star, ID (US)

(72) Inventor: Kristy Rowberry Martin, Star, ID (US)

(21) Appl. No.: 15/019,932

(22) Filed: Feb. 9, 2016

Related U.S. Application Data
(60) Provisional application No. 62/114,463, filed on Feb. 10, 2015.

(51) Int. Cl. A61M 16/08; A61M 16/06

(52) U.S. Cl. CPC A61M 16/0816; A61M 16/06; A61M 16/0891

(57) ABSTRACT

A respiration insert includes a first portion sized to insert within an aperture of an induction mask formerly occupied by an anesthesia circuit. The respiration insert further includes a second portion coupled to the first portion and defining ports in fluid communication with a channel defined by the first portion. The ports may be embodied as tube stubs and may include an attachment interface for coupling to a CO₂ monitoring system. A flange extends outwardly from the respiration insert and defines slots for receiving straps used to secure the respiration insert and induction mask to the head of a patient.

### PRIORITY CLAIM

This application claims the benefit of U.S. Provisional Application Ser. No. 62/114,463 filed Feb. 10, 2015 and entitled DABILATOR, which is hereby incorporated herein by reference in its entirety.

### FIELD OF THE INVENTION

This invention relates to ventilation and anesthesia delivery systems and methods.

### BACKGROUND OF THE INVENTION

A typical operation will include the use of an induction mask coupled to an anesthesia circuit. Anesthesia is delivered through the induction mask prior to and during an operation. The induction mask is then removed and replaced with a so-called “simple mask” that delivers respiration gasses during transport to a recovery room.

The apparatus disclosed herein provides an improved approach for providing post-operation ventilation.

### SUMMARY OF THE INVENTION

In one aspect of the invention, an apparatus is disclosed for use with an induction mask defining a flexible shroud adapted to cover a nose and mouth of a patient and an aperture adapted to and secured to an anesthesia circuit. The apparatus includes a first portion defining a central channel and sized to insert within the aperture and be elastically restrained by the aperture. A second portion defines first and second ports and is coupled to the first portion, the first and second ports being in fluid communication with the central channel. The apparatus further includes first and second strap receivers coupled to the second portion.

In some embodiments, the first portion is cylindrical and defines a central axis. The apparatus may further include a flange positioned between the first and second portions and extending outwardly perpendicular to the central axis. In some embodiments, the first and second strap receivers are formed on the flange. The first and second strap receivers may include first and second slots defined in the flange.

In some embodiments, the first and second ports include first and second tube stubs secured to the second portion and defining first and second channels in fluid communication with the central channel. In some embodiments, the central channel defines a central axis and the first and second channels are parallel with one another and with the central axis.

In some embodiments, the second portion defines a planar outer surface perpendicular to the central axis, the first and second tube stubs protruding from the planar outer surface. In some embodiments, an exhaust aperture extends through the planar surface and is in fluid communication with the central channel. In order to fit within standard induction masks, the first portion may be cylindrical and have an outer diameter of 0.87 inches. Also, in the preferred embodiment, the internal diameter of the first portion is sized to receive a child induction mask (0.625 inches).

In some methods of use, an anesthesia circuit is removed from the aperture of the induction mask and replaced with the respirator insert as recited above. A strap is engaged with the first and second strap receivers and is passed over a head of the patient such that the strap retains the induction mask and respirator insert over the nose and mouth of the patient. The first and second strap receivers may include first and second slots defined in the flange and the strap may be inserted within the first and second slots.

In some methods of use, a carbon dioxide monitoring system is coupled to the second port. In some embodiments, the second portion further defines a third port in fluid communication with the central channel and coupling the central channel to the atmosphere. The method may therefore include venting expiration gasses of the patient through the third port.

### BRIEF DESCRIPTION OF THE DRAWINGS

Preferred and alternative examples of the present invention are described in detail below with reference to the following drawings:

FIG. 1 is an isometric view of a respiration insert for an induction mask in accordance with an embodiment of the present invention;

FIG. 2 is a top view of the respiration insert of FIG. 1;

FIG. 3 is a cross-sectional elevational view of the respiration insert of FIG. 1 along line A-A;

FIG. 4 is a cross-sectional elevational view of the respirator insert of FIG. 1 along line B-B;

FIG. 5 is a partial cross-sectional view of the flange of the respiration insert of FIG. 1;

FIG. 6 is a bottom view of the respiration insert of FIG. 1;

FIG. 7 is a side view of an induction mask in accordance with the prior art;

FIG. 8 is a top view of the induction mask of FIG. 7;

FIG. 9 is an isometric view of an alternative embodiment of a respiration insert in accordance with an embodiment of the present invention;

FIG. 10 is a side view of the respiration insert of FIG. 9;

FIG. 11 is a top view of the respiration insert of FIG. 9;

FIG. 12 is a cross-sectional view of the respiration insert of FIG. 9 along the line C-C;

FIG. 13 is another isometric view of the respiration insert of FIG. 9; and

FIG. 14 illustrates an induction mask having a respiration insert in use with a patient in accordance with an embodiment of the present invention.

### DETAILED DESCRIPTION OF THE PREFERRED EMBODIMENT

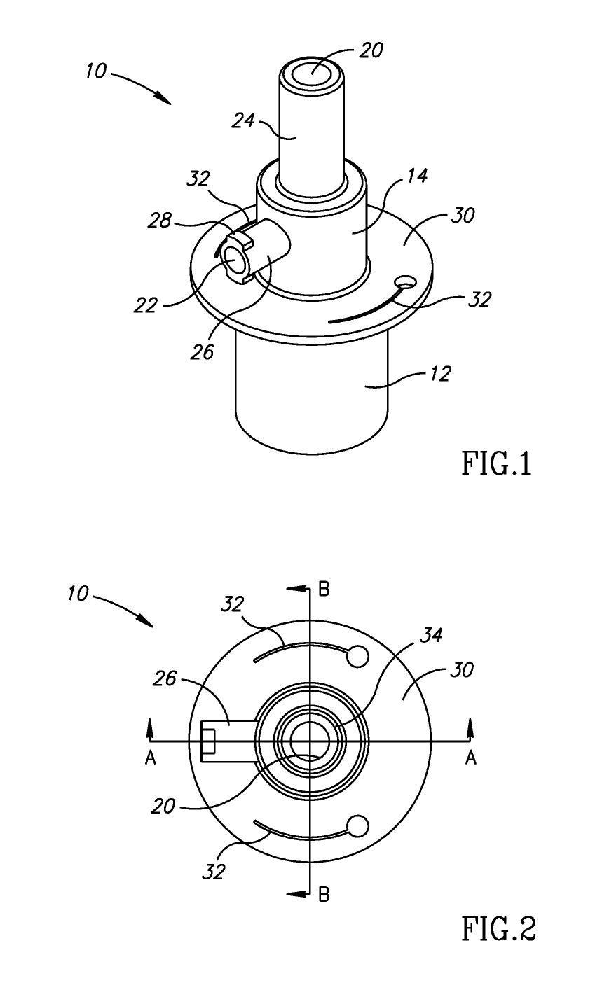

Referring to FIGS. 1 and 2 a respiration insert 10 may be used in combination with an induction mask that has previously been used for delivery of anesthesia. The insert 10 may include a first portion 12 sized to insert within an aperture in the induction mask and be elastically retained within the aperture. The insert 10 further includes a portion 14 positioned outside of the induction mask when the first portion 12 is inserted within the aperture.

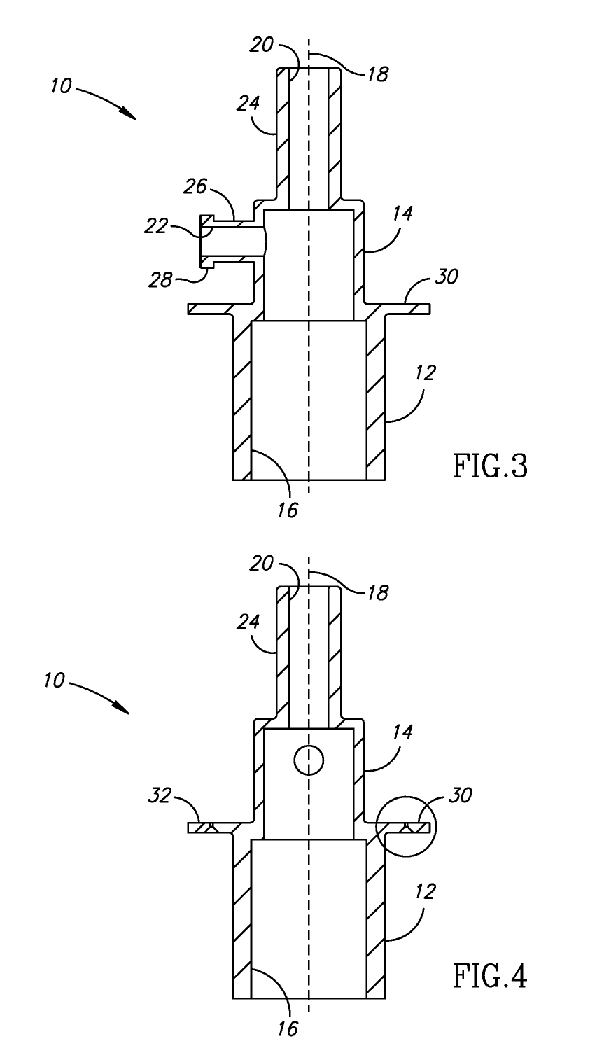

FIG. 3 illustrates the respiration insert 10 sectioned along line A-A shown in FIG. 2 and FIG. 4 illustrates the respiration insert 10 sectioned along line B-B. The first portion 12 and possibly the portion 14 define a central channel 16, which may be cylindrical about a central axis 18. The portions 12, 14 may also be cylindrical about the central axis 18. A first port 20 and a second port 22 are defined on the portion 14. In the illustrated embodiment, the first port 20 is defined by a tube stub 24 protruding from the portion 14. The tube stub 24 may by cylindrical about the central axis 18 and is in fluid communication with the central channel 16. The tube stub 24 may be sized to insert within or receive a tube for delivering oxygen or other mix of inhalation gasses to a patient.

The second port 22 may be defined by a tube stub 26 likewise protruding from the portion 14 and in fluid communication with the central channel 16. In the illustrated embodiment, the tube stub 26 is cylindrical with the axis thereof perpendicular to the central axis 18. The tube stub 26 may define an attachment interface 28 (e.g. twist lock, friction fit, or the like) for securement to any known gas measurement or delivery system. For example, the port 22 may couple to a system for measuring end tidal CO₂ (etCO₂), such as in the context of monitored anesthesia care (MAC), an adjunct to the distal end of a laryngeal mask airway (LMA), with an endotracheal tube or tracheostomy cannula for transport, or for intravenous anesthetic (TIVA) cases. In such applications, the use of the respiratory insert 10 reduces the need and corresponding expense of an anesthetic circuit specifically for TIVA cases.

The respiratory insert 10 further includes a flange 30 that protrudes outwardly from the first portion 12 and second portion 14 and is positioned between the first portion 12 and second portion 14. In particular, the flange is a disk that radiates outwardly from the central axis 18 such that the upper and lower surfaces of the flange 30 are perpendicular to the central axis 18. The flange 30 may have slots 32 formed therein in order to receive straps for securing the combination of the respiratory insert 10 and an induction mask to the face of a patient.

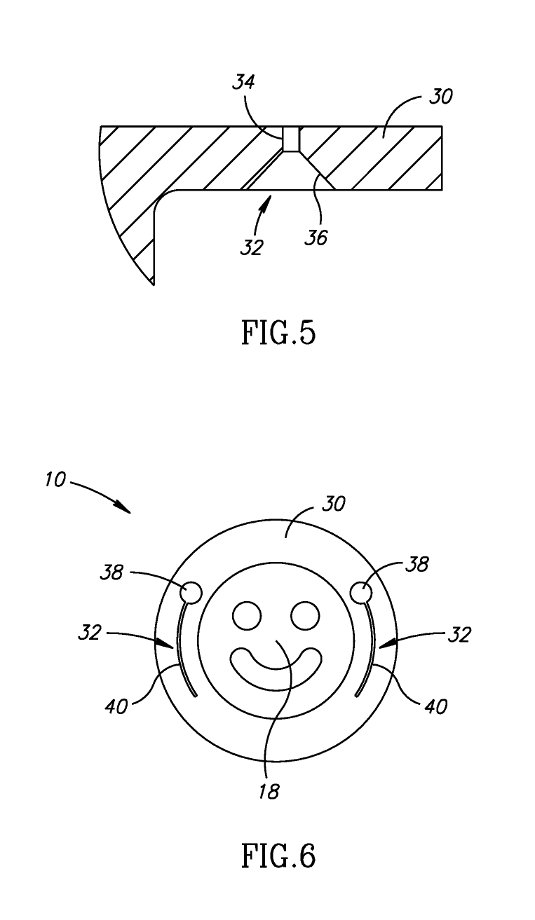

Referring to FIGS. 5 and 6, in the illustrated embodiment, the slots 32 include a straight portion 34 adjacent a first face of the flange 30 and a flared portion adjacent a second face opposite the first face of the flange 30. The slots 32 each further include a wide entry portion 38 for receiving insertion of straps and a narrow portion 40. The narrow portion of the slots 32 may be arcuate in shape with a radius of curvature centered on the central axis 18. In use, an end of a strap is inserted through the widened portion 38, tied into a knot, and then slid into the narrow portion 40 such that the knot will prevent removal of the strap.

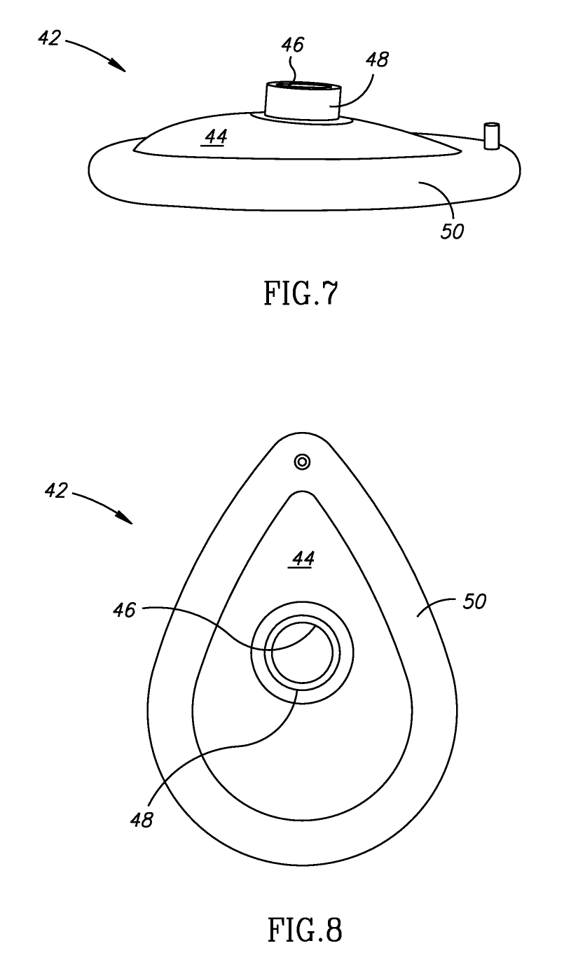

Referring to FIGS. 7 and 8, the respiration insert 10 may be used with the illustrated induction mask 42. The induction mask 42 may be any type or brand of induction mask known in the art. The typical induction mask 42 includes a flexible shroud 44 sized to cover the nose and mouth of a patient. Different sizes of induction masks 42 may exist with shrouds of different sizes to accommodate different patients, e.g. adult vs. child patients.

An aperture 46 may extend through the shroud 44 and may be defined by a tube stub 48 protruding from the shroud 44. The first portion 12 of the insert 10 is sized to insert within the aperture 46. In particular, the aperture 46 may be sized such that force is required to insert the first portion 12 within the aperture 46 such that friction maintains the first portion 12 within the aperture 46. In many instances, the size of the aperture 46 is uniform for various sizes of induction masks 42. Accordingly, the respiration insert 10 may be used for patients of various sizes. In some embodiments, the first portion 12 has a cylindrical surface with a diameter of 0.87 inches in order to insert within a standard-sized aperture 46. Also, in the preferred embodiment, the internal diameter of the first portion 12 is sized to receive a child induction mask (0.625 inches or 15 mm).

In some embodiments, the induction mask may include a rim 50 surrounding the shroud 55. The rim 50 may be more or less rigid than the shroud 44 and may further include folded or rounded shape such that the rim 50 provides a smooth and deformable surface in contact with the face of a patient, thereby providing a degree of sealing.

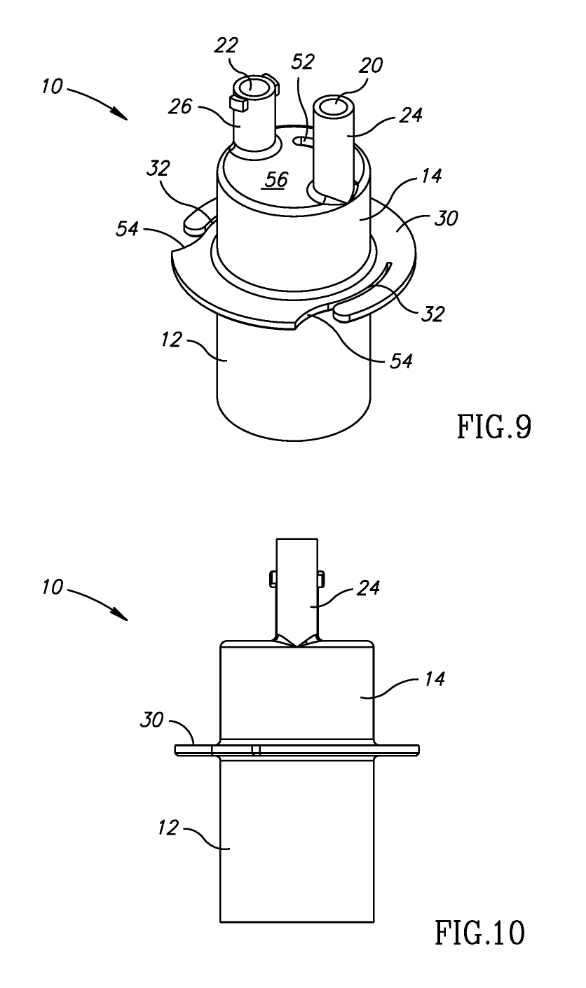

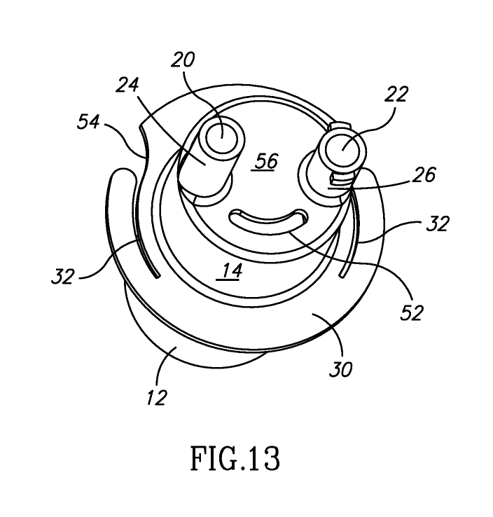

Referring to FIGS. 9 through 13, the respiration insert 10 may have various alternative configurations. For example, in addition to the ports 20, 22, an additional exhaust port 52 may be defined on the second portion 14. The exhaust port 52 may not provide for an attachment of a tube or other device. Accordingly, the exhaust port 52 may simply be an aperture through the second portion 14 in fluid communication with the central channel 16.

The slots 32 may likewise have an alternative configuration. For example, the slots may include a flared portion 54 that extends to the edge of the flange 30, thereby enabling a strap to slide readily into the slots 32. The second portion 14 may include a planar outer face 56 that is perpendicular to the central axis 18. The tube stubs 24, 26 may protrude from this face 56. The exhaust port 52 may likewise pass through the planar outer face 56.

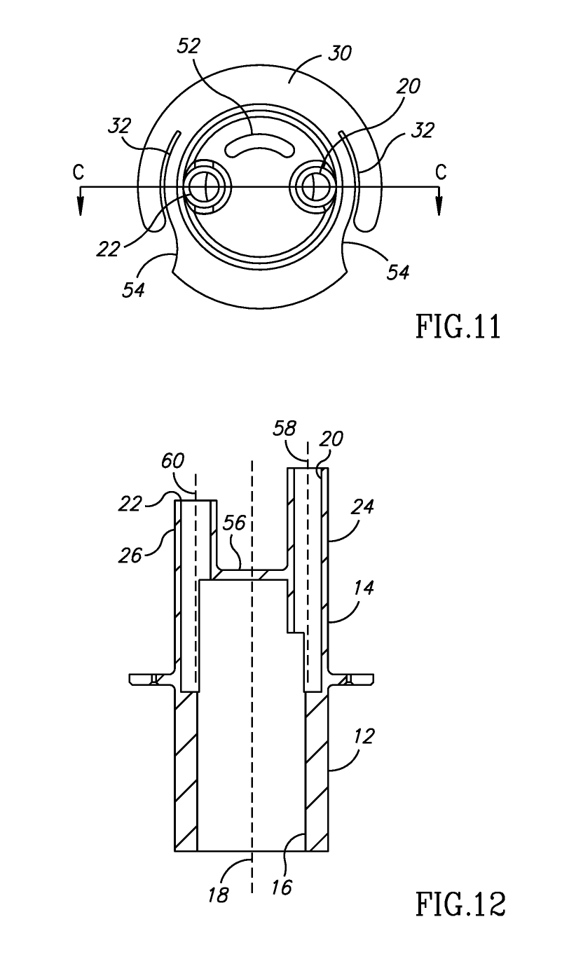

FIG. 12 illustrates a cross-section of the respiration insert along line C-C shown in FIG. 11. The axes 58, 60 of the tube stubs 24, 26 may be parallel to the central axis 18. and the channels of the tube stubs 24, 26 intersect the central channel 16 thereby enabling flow of gasses. As shown in FIG. 11, the arrangement of the exhaust port 52 and tube stubs 34, 26 may be arranged decoratively, such as in smiley face or other arrangement.

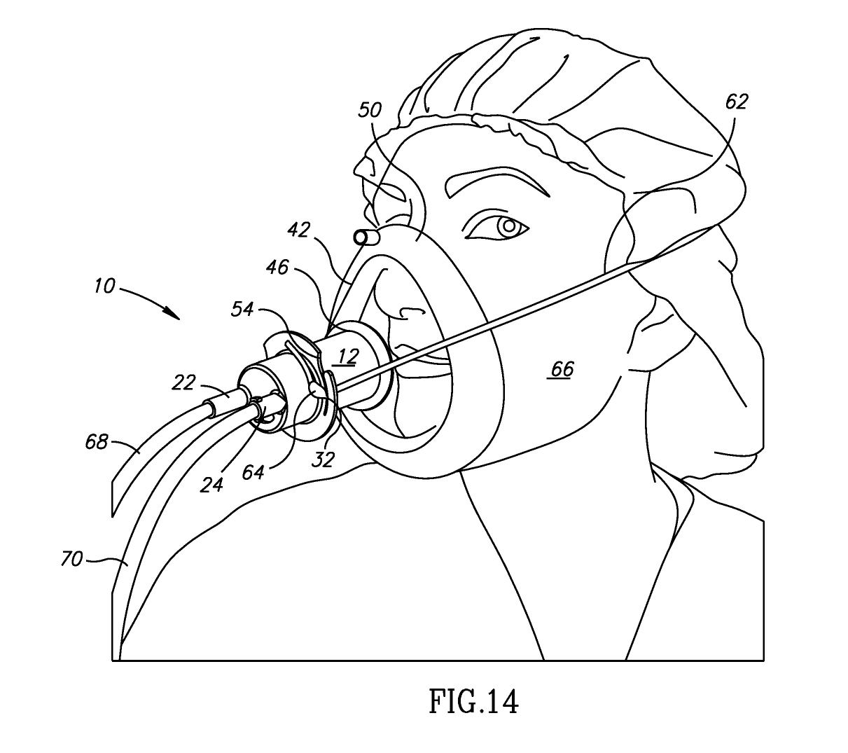

FIG. 14 illustrates the respiratory insert in use with an induction mask 42. The first portion 12 is inserted into the aperture 46. In each slot 32, an end of a strap 62 is inserted. In particular, a knot 64 is tied at each end of the strap 62 and the strap 62 is inserted into the slot 32, such that the slot 32 is positioned between the knot 64 and the head 66 of the patient. The strap is stretched around the head 66 of the patient such that the elasticity of the strap 62 holds the respiratory insert 10 and induction mask 42 against the face of the patient. The use of slots 32 with a strap 62 is just one example of how straps may be used to secure the respiration insert 10 and induction mask 42 to a patient's face.

One or more tubes 68, 79 may be attached to the tube stubs 24, 26. Where only oxygen delivery is needed, only one tube stub 24 will have a tube 68 secured thereto. Where measurement of expiration gasses (e.g. etCO₂) is needed, a second tube 70 may be secured to the other tube stub 26. In a typical method of use, an anesthesia circuit is removed from the aperture 46 and the respiration insert 10 is put in its place as shown in FIG. 14. In this manner a separate mask is not required for respiration, thereby reducing waste.

Various modifications of the respiration insert 10 may be made. For example, the ports 20, 22 and corresponding tube stubs 24, 26 may be different sizes. As shown above, the orientations of the tube stubs 24, 26 may be the same, e.g. parallel, or different, e.g. perpendicular or at some other angle relative to one another. The tube stubs 24, 26 may protrude both outside of the second portion 14 and may also protrude into the central channel 16. Alternatively, the tube stubs 24, 26 may terminate at the central channel 16. The tube stubs 24, 26 may be different colors then the first portion 12 or second portion 14 in order to facilitate identification.

As is apparent above, the exhaust port 52 has an arcuate shape that, with the ports 20, 22, defines a smiley face. The arrangement and shape of the exhaust port 52 and ports 20, 22 may be different in order to provide a different decorative design.

As is apparent above, various configurations of the slots 32 are possible. In the illustrated embodiment, there are two slots 32 and a single strap 62 that passes around the head of a patient. In some embodiments, two straps 62 are used that pass around the ears of a patient. In such embodiments, four slots 32 may be use, with pairs of slots 32 on opposite sides of the second portion 14. In that manner each pair of slots 32 may engage the ends of one of the two straps 62.

In some embodiments, the central channel 16 may be coated with or otherwise treated with a scented substance, such as an essential oil in order to make the use of the respiration insert 10 more pleasant.

The respiration insert 10 in combination with an induction mask 42 may be used for various medical applications. For example, the respiration insert 10 may be used with the induction mask 42 to provide respiration during transportation, thereby improving the safety and comfort of the patient. The respiration insert 10 and induction mask 42 may be used during monitored anesthesia care (MAC) case to permit etCO2 monitoring. The respiration insert 10 in combination with the induction mask may be used as an adjunct to the distal end of a laryngeal mask airway (LMA) or an endotracheal tube or a tracheostomy cannula for transport and for total intravenous anesthetic (TIVA) cases. TIVA cases are the general anesthetic of choice in cases where nerve monitoring is required or if the patient has or has a family history of Malignant Hyperthermia. These applications would reduce the need/cost of an anesthetic circuit specifically for TIVA cases.

In all of the above medical application, hospital costs are reduced by using the combined respiration insert 10 and induction mask 42. For example, use of the respiration insert 10 and induction mask 42:
Eliminates the cost and storage requirements for a separate “simple mask” during patient transport after an operation;
Eliminates the need to use a altered nasal cannula in the recovery room (the respiration insert 10 can be utilized in the recovery room to monitor respirations via the etCO2 port 22);
Decreases carbon credit usage (currently, the induction mask 42 and any circuits are thrown away after use in the operating room); and
Decreases the need of large storage space (no need for storage of simple masks or as many circuits).

The use of the respiration insert and induction mask further enhances patient comfort and safety:
It allows O₂ delivery and end tidal CO₂ (etCO₂) monitoring. (Monitoring etCO2 is vital when assessing patient ventilation status).
It permits reuse the induction mask 42 with the respiration insert 10 assures that the induction mask is readily available throughout transport and in the recovery room (the induction mask 42 is critical when a patient needs to be manually ventilated).
The induction mask 42 is more comfortable for patients compared to the simple mask. Kids induction masks 42 also smell better as flavored lip balm may be placed in the induction the masks 42.
The simple mask is more prone to causing corneal abrasions, does not allow adequate assessment of patient ventilation, does not enable ventilation of patients in the emergency event of apnea, is uncomfortable, tends to shift up on the face thereby covering the patient's eyes and blowing oxygen in them.

While the preferred embodiments of the invention have been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. An apparatus for use with an induction mask defining a flexible shroud adapted to cover a nose and mouth of a patient and an aperture adapted to secured to an anesthesia circuit, the apparatus comprising:
a first portion defining a central channel and sized to insert within the aperture and be elastically restrained by the aperture; and
a second portion defining first and second ports coupled to the first portion, the first and second ports being in fluid communication with the central channel.

2. The apparatus of claim 1, further comprising first and second strap receivers coupled to the second portion.

3. The apparatus of claim 2, wherein the first portion is cylindrical and defines a central axis, the apparatus further comprising a flange positioned between the first and second portions and extending outwardly perpendicular to the central axis.

4. The apparatus of claim 3, wherein the first and second strap receivers are formed on the flange.

5. The apparatus of claim 4, wherein the first and second strap receivers include first and second slots defined in the flange.

6. The apparatus of claim 1, wherein the first and second ports include first and second tube stubs secured to the second portion and defining first and second channels in fluid communication with the central channel.

7. The apparatus of claim 6, wherein the central channel defines a central axis and the first and second channels are parallel with one another and with the central axis.

8. The apparatus of claim 7, wherein the second portion defines a planar outer surface perpendicular to the central axis, the first and second tube stubs protruding from the planar outer surface.

9. The apparatus of claim 8, further comprising an exhaust aperture extending through the planar surface and in fluid communication with the central channel.

10. The apparatus of claim 1, wherein the first portion is cylindrical and has an outer diameter sized to fit a standard induction mask and an inner diameter sized to fit a standard child induction mask.

11. A method comprising:
providing an induction mask defining a flexible shroud adapted to cover a nose and mouth of a patient and an aperture having an anesthesia circuit connector inserted therein;
removing the anesthesia circuit connector from the aperture;
inserting a respirator insert into the aperture, the respirator insert including
a first portion defining a central channel positioned within the aperture and elastically restrained by the aperture; and
a second portion defining first and second ports coupled to the first portion, the first and second ports being in fluid communication with the central channel; and
coupling an oxygen source to the first port.

12. The method of claim 11, wherein the respirator insert includes first and second strap receivers coupled to the second portion and a strap engaging the first and second strap receivers, the method further comprising passing the strap over a head of the patient such that the strap retains the induction mask and respirator insert over the nose and mouth of the patient.

13. The method of claim 12, wherein the first portion is cylindrical and defines a central axis, the apparatus further comprising a flange positioned between the first and second portions and extending outwardly perpendicular to the central axis, the first and second strap receivers being formed on the flange.

14. The method of claim 13, wherein the first and second strap receivers include first and second slots defined in the flange, the method further comprising inserting the strap within the first and second slots.

15. The method of claim 11, further comprising coupling a carbon dioxide monitoring system to the second port.

16. The method of claim 15, wherein the second portion further defines a third port in fluid communication with the central channel and coupling the central channel to the atmosphere, the method further comprising venting expiration gasses of the patient through the third port.

17. The method of claim 16, wherein the first and second ports include first and second tube stubs secured to the second portion and defining first and second channels in fluid communication with the central channel.

18. The method of claim 17, wherein the central channel defines a central axis and the first and second channels are parallel with one another and with the central axis.

19. The method of claim 18, wherein the second portion defines a planar outer surface perpendicular to the central axis, the first and second tube stubs protruding from the planar outer surface.

20. The method of claim 19, wherein the third port extends through the planar surface.
